(19) United States
(12) Patent Application Publication
(10) Pub. No.: US 2026/0015577 A1
(43) Pub. Date: Jan. 15, 2026

(54) COMPOSITION FOR 3D PRINTING SUPPORT OR 3D CELL CULTURE SUPPORT

(71) Applicant: JSR CORPORATION, Tokyo (JP)

(72) Inventors: Minato AKIYAMA, Minato-ku (JP); Kunihiko KOBAYASHI, Minato-ku (JP)

(73) Assignee: JSR CORPORATION, Tokyo (JP)

(21) Appl. No.: 18/881,994

(22) PCT Filed: Jul. 5, 2023
(86) PCT No.: PCT/JP2023/024928

§ 371 Date: Jan. 7, 2025

(30) Foreign Application Priority Data
Jul. 12, 2022 (JP) 2022-111832

(51) Int. Cl. C12N 5/00; B29C 64/112; B29C 64/40; B29L 31/00; B33Y 10/00; C08F 226/02

(52) U.S. Cl. CPC C12N 5/0062; B29C 64/112; B29C 64/40; B33Y 10/00; C08F 226/02; B29L 2031/753; C08F 2800/20; C08F 2810/20; C08F 2810/50

(57) ABSTRACT

A composition may be useful for a 3D printing support or a 3D cell culture support and be excellent in salt tolerance, including a (A) a polymer having a structural unit of formula (1)

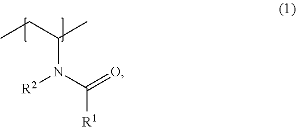

R¹ and R² independently being a hydrogen atom or an alkyl group having 1 to 10 carbon atoms, or R¹ and R² together optionally forming a ring structure having 3 to 10 carbon atoms; and (B) an aqueous medium.

### TECHNICAL FIELD

The present invention relates to a composition for 3D printing support or 3D cell culture support.

### BACKGROUND ART

3D printing refers to three-dimensional modeling based on 3D model data, and 3D bioprinting for producing, for example, a three-dimensional cell pattern using this technology has attracted attention in the field of regenerative medicine.

In order to use the three-dimensional cell pattern as a biological tissue or an organ, it is necessary that the three-dimensional cell pattern is complicated and flexible similarly to an actual biological tissue or an organ. In order to produce such a pattern, 3D bioprinting is performed using a support (support exhibiting Bingham plastic behavior) that exhibits a liquid-like behavior at the time of stress application (at the time of drawing) and exhibits a solid-like behavior when no stress is applied after drawing (Patent Literature 1).

### CITATION LIST

### Patent Literature

Patent Literature 1: WO2018/187595
Patent Literature 2: WO2018/165584

### SUMMARY OF INVENTION

### Technical Problem

Meanwhile, a 3D cell culture support using anionic microgel particles derived from methacrylic acid or the like is known, and 3D bioprinting using this support has also been proposed (Patent Literature 2).

However, it found that the support described in Patent Literature 2 has insufficient salt tolerance, and when a salt or ions are added, charge shielding or chelation occurs, resulting in lowering the viscosity and thus deteriorating the shape maintainability of a support. In addition, it found that when the support is used as a 3D cell culture support, it is difficult for components necessary for culture, such as calcium ions, to reach the cells. It found that it is difficult to use ink that is cured in response to calcium ions for 3D printing, for example.

An object to be solved by the present invention is to provide a composition that is useful for a 3D printing support or a 3D cell culture support, and is excellent in salt tolerance.

### Solution to Problem

The object has been achieved by the following means <1> to <14>.
<1> A composition for a 3D printing support or a 3D cell culture support, including the following component (A) and component (B) (hereinafter, also referred to as a composition of the present invention or a composition for a 3D printing support or a 3D cell culture support of the present invention).
(A) Polymer having a structural unit represented by Formula (1)
(B) Aqueous medium

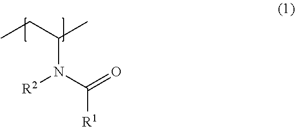

[In Formula (1), R¹ and R² independently represent a hydrogen atom or an alkyl group having 1 to 10 carbon atoms, or R¹ and R² together may form a ring structure having 3 to 10 carbon atoms.]
<2> The composition according to <1>, in which the polymer further has a structural unit represented by Formula (2).

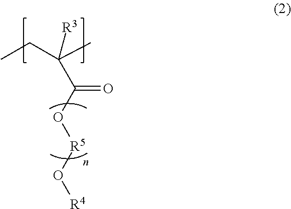

[In Formula (2), R³ and R⁴ independently represent a hydrogen atom or a methyl group, R⁵ represents an alkylene group having 2 to 4 carbon atoms, and n represents 1 to 1000 in terms of an average value.]
<3> The composition according to <1> or <2>, in which the polymer further has a structural unit derived from a crosslinkable monomer.
<4> The composition according to <3>, in which the structural unit derived from the crosslinkable monomer is one or more selected from the group consisting of a structural unit derived from a vinyl-based crosslinkable monomer, a structural unit derived from an allyl-based crosslinkable monomer, a structural unit derived from a (meth)acrylate-based crosslinkable monomer, and a structural unit derived from a (meth)acrylamide-based crosslinkable monomer.
<5> The composition according to any one of <1> to <4>, in which the polymer is a particulate polymer.
<6> The composition according to any one of <1> to <5>, in which a ratio (X/Y) between a viscosity X measured under conditions of a measurement temperature of 23° C. and a shear rate of 0.1 sec⁻¹ using a rotary viscometer and a viscosity Y measured under conditions of a measurement temperature of 23° C. and a shear rate of 100 sec⁻¹ using a rotary viscometer is 10 or more.
<7> The composition according to any one of <1> to <6>, in which a viscosity change rate when NaCl is added, calculated in accordance with Formula (α), is 20% or less.

$$Viscosity change rate ( \% ) = \{ ( Viscosity before addition of NaCl ) - ( Viscosity after addition of NaCl ) \} / ( Viscosity before addition of NaCl ) \times 100 \qquad ( α )$$

[In Formula (α), the viscosity before addition of NaCl means a viscosity (mPa·s) of the composition when measured under the conditions of a measurement temperature of 23° C. and a shear rate of 100 sec⁻¹ using a rotary viscometer, and the viscosity after addition of NaCl means a viscosity (mPa·s) of a composition, which is obtained by adding NaCl to the composition so that the resulting NaCl concentration is 0.15 mol/L and then allowing the composition to stand for 60 minutes and which is measured under the conditions of a measurement temperature of 23° C. using a rotary viscometer.]
<8> A composition for a 3D printing support or a 3D cell culture support, including the following component (A2) and component (B) (hereinafter, also referred to as a composition (II) of the present invention).
(A2) Nonionic polymer in which a pure water content at which a viscosity of a dispersion reaches a value less than 1000 mPa·s when the viscosity of the dispersion is measured using a rotary viscometer under the conditions of a measurement temperature of 23° C. and a shear rate of 100 sec⁻¹ while dispersing the dispersion in pure water is 70 mass % or more
(B) Aqueous medium
<9> A method for producing a three-dimensional structure, including a step (i) and a step (ii) (hereinafter, also referred to as a three-dimensional structure production method of the present invention).
(i) Step of filling a container with the composition according to any one of <1> to <8>
(ii) Step of bringing a second composition into contact with the composition with which the container is filled in step (i)
<10> The production method according to <9>, in which the step (ii) is to inject the second composition into the composition with which the container is filled in step (i), while applying shear.
<11> The production method according to <9> or <10>, in which the second composition contains a cell and an aqueous medium.
<12> The production method according to <11>, in which the second composition further contains an extracellular matrix.
<13> The production method according to any one of <9> to <12>, in which the three-dimensional structure is an organoid or a spheroid.
<14> A three-dimensional structure obtained by the production method according to any one of <9> to <13>.

### Advantageous Effects of Invention

The composition of the present invention is useful for a 3D printing support or a 3D cell culture support and is excellent in salt tolerance.

### BRIEF DESCRIPTION OF DRAWINGS

FIG. 1 is a view showing a test pattern in a case where a composition for a support of Example 1 is used.

FIG. 2 is a view showing a test pattern in a case where a composition for a support of Comparative Example 1 is used.

### DESCRIPTION OF EMBODIMENTS

### (Composition)

A composition for a 3D printing support or a 3D cell culture support of the present invention includes a component (A) and a component (B) as follows.
(A) Polymer having a structural unit represented by Formula (1)
(B) Aqueous medium

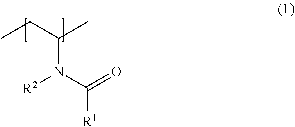

[In Formula (1), R¹ and R² independently represent a hydrogen atom or an alkyl group having 1 to 10 carbon atoms, or R¹ and R² together may form a ring structure having 3 to 10 carbon atoms.]

### (Component (A))

The composition of the present invention contains (A) a polymer having a structural unit represented by Formula (1). Due to such a component (A) being contained, excellent salt tolerance can be obtained while satisfying 3D printing performance requirements and ease of three-dimensional cell culture.

In Formula (1), the number of carbon atoms of the alkyl group represented by R¹ and R² is preferably 1 to 8, more preferably 1 to 4, and particularly preferably 1 or 2 in order to obtain a desired viscosity at the time of stress application while satisfying having salt tolerance. The alkyl group may be linear or branched. Examples of the alkyl group include a methyl group, an ethyl group, a n-propyl group, an isopropyl group, a n-butyl group, a sec-butyl group, a tert-butyl group, a pentyl group, a hexyl group, a heptyl group, an octyl group, a nonyl group, and a decyl group.

In addition, R¹ is preferably a hydrogen atom or an alkyl group having 1 to 10 carbon atoms, more preferably a hydrogen atom or an alkyl group having 1 to 8 carbon atoms, still more preferably a hydrogen atom or an alkyl group having 1 to 4 carbon atoms, and particularly preferably a hydrogen atom or an alkyl group having 1 or 2 carbon atoms, in order to obtain a desired viscosity at the time of stress application while satisfying having salt tolerance. R² is preferably a hydrogen atom in order to obtain a desired viscosity at the time of stress application.

The number of carbon atoms of a ring structure formed by bonding R¹ and R² to each other is preferably 4 to 8 and more preferably 4 to 6.

A structural unit (1) in a case where R¹ and R² together form a ring structure having 3 to 10 carbon atoms is preferably at least one selected from the group consisting of a structural unit represented by the following Formula (1-1), a structural unit represented by the following Formula (1-2), and a structural unit represented by the following Formula (1-3), and is particularly preferably a structural unit represented by the following Formula (1-1).

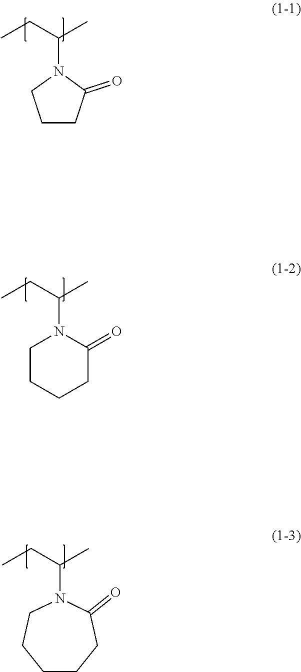

Examples of a monomer from which the structural unit (1) is derived include N-vinylformamide, N-vinylacetamide, N-vinylpropionamide, N-vinylbutylamide, N-vinylisobutylamide, N-vinyl-2-methylbutanamide, N-vinyl-3-methylbutanamide, N-vinyl-2,2-dimethylpropionamide, N-vinylvaleramide, N-methyl-N-vinylformamide, N-ethyl-N-vinylformamide, N-propyl-N-vinylformamide, N-isopropyl-N-vinylformamide, N-methyl-N-vinylacetamide, 1-vinyl-2-pyrrolidone, 1-vinyl-2-piperidone, and N-vinylcaprolactam. These monomers can be used singly or in combination of two or more kinds thereof.

Among them, N-vinylacetamide, N-vinylformamide, 1-vinyl-2-pyrrolidone, and N-vinylpropionamide are preferable, and N-vinylacetamide is particularly preferable.

The content ratio of the structural unit (1) is preferably 50 mass % or more, more preferably 55 mass % or more, and particularly preferably 64 mass % or more with respect to all structural units in the polymer of the component (A) in order to obtain a desired viscosity at the time of stress application while satisfying having salt tolerance, to improve printing performance, and to improve ease of culture, and is preferably 95 mass % or less, more preferably 90 mass % or less, and particularly preferably 85 mass % or less with respect to all structural units in the polymer of the component (A) in order to obtain desired viscosity characteristics before and after application of stress and satisfy having ease of production. A specific range thereof is preferably 50 mass % or more and 95 mass % or less, more preferably 55 mass % or more and 90 mass % or less, and particularly preferably 64 mass % or more and 85 mass % or less with respect to all structural units in the polymer of the component (A).

In a case where the content ratio of the structural unit (1) is 64 mass % or more, printing performance and ease of culture are particularly favorable.

The content ratio of the structural unit (1) can be measured by pyrolysis gas chromatography-mass spectrometry (PyGC-MS), CHN elemental analysis, ¹H-NMR, ¹³C-NMR, or the like.

The polymer of the component (A) preferably further has a structural unit represented by Formula (2) in addition to the structural unit (1) in order to obtain desired viscosity characteristics before and after application of stress by adjusting interaction between polymer molecules and to satisfy having ease of production.

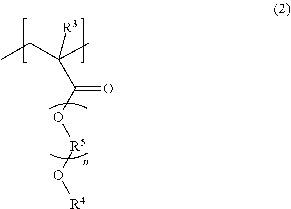

[In Formula (2), R³ and R⁴ independently represent a hydrogen atom or a methyl group, R⁵ represents an alkylene group having 2 to 4 carbon atoms, and n represents 1 to 1000 in terms of an average value.]

R⁴ is preferably a methyl group in order to obtain desired viscosity characteristics before and after application of stress and to satisfy having ease of production.

R⁵ represents an alkylene group having 2 to 4 carbon atoms, and n R⁵'s may be the same as or different from each other.

The number of carbon atoms of the alkylene group represented by R⁵ is preferably 2 or 3, and more preferably 2.

Also, the alkylene group represented by R⁵ may be linear or branched. Examples of the alkylene group include an ethane-1,2-diyl group, a propane-1,2-diyl group, a propane-1,3-diyl group, a propane-2,2-diyl group, a butane-1,2-diyl group, a butane-1,3-diyl group, and a butane-1,4-diyl group. Among them, an ethane-1,2-diyl group is preferable.

n represents 1 to 1000 in terms of an average value, and is preferably 2 or more in terms of an average value, more preferably 4 or more in terms of an average value, still more preferably 8 or more in terms of an average value, and particularly preferably 10 or more in terms of an average value, in order to obtain desired viscosity characteristics before and after application of stress, to improve printing performance, to enhance ease of culture, and to satisfy having ease of production, and is preferably 500 or less in terms of an average value, more preferably 250 or less in terms of an average value, still more preferably 100 or less in terms of an average value, still further preferably 50 or less in terms of an average value, and particularly preferably 35 or less in terms of an average value in order to obtain desired viscosity characteristics before and after application of stress and to improve printing performance and to satisfy having ease of production. Specifically, in order to obtain desired viscosity characteristics before and after application of stress, to improve printing performance, to enhance ease of culture, and to satisfy having ease of production, n is preferably 2 to 500 in terms of an average value, more preferably 4 to 250 in terms of an average value, still more preferably 8 to 100 in terms of an average value, still further preferably 10 to 50 in terms of an average value, and particularly preferably 10 to 35 in terms of an average value.

In a case where n in Formula (2) is 35 or less in terms of an average value, the printing performance and the ease of culture are particularly favorable.

Each “average value” in the present specification can be measured by NMR. For example, in a case where R⁴ in Formula (2) is a methyl group, an average value of n can be calculated by measuring ¹H-NMR for the structure of Formula (2) and comparing integral values of proton peaks of an alkylene group having 2 to 4 carbon atoms represented by R⁵ and a methyl group represented by R⁴.

Also, in a case where the content ratio of the structural unit (1) is 64 mass % or more and n in Formula (2) is 35 or less in terms of an average value, particularly in a case where the content ratio of the structural unit (1) is 64 mass % or more and 85 mass % or less and n in Formula (2) is 10 to 35 in terms of an average value, the printing performance and the ease of culture are particularly favorable.

Examples of the monomer from which the structural unit (2) is derived include ethylene glycol mono(meth)acrylate, diethylene glycol mono(meth)acrylate, triethylene glycol mono(meth)acrylate, polyethylene glycol mono(meth)acrylate, 2-methoxyethyl(meth)acrylate, methoxydiethylene glycol (meth)acrylate, methoxytriethylene glycol (meth)acrylate, methoxypolyethylene glycol (meth)acrylate, polypropylene glycol mono(meth)acrylate, and methoxypolypropylene glycol (meth)acrylate. These monomers can be used singly or in combination of two or more kinds thereof.

The content ratio of the structural unit (2) is preferably 5 mass % or more, more preferably 7 mass % or more, and particularly preferably 10 mass % or more with respect to all the structural units in the polymer of the component (A) in order to satisfy having ease of production while obtaining desired viscosity characteristics before and after application of stress, and is preferably 50 mass % or less, more preferably 40 mass % or less, and particularly preferably 30 mass % or less with respect to all the structural units in the polymer of the component (A) in order to improve printing performance, to enhance ease of culture, to obtain a sufficient viscosity, and to satisfy shape maintainability of a three-dimensional structure. A specific range thereof is preferably 5 mass % or more and 50 mass % or less, more preferably 7 mass % or more and 40 mass % or less, and particularly preferably 10 mass % or more and 30 mass % or less with respect to all structural units in the polymer of the component (A).

In a case where the content ratio of the structural unit (2) is 30 mass % or less, printing performance and ease of culture are particularly favorable.

The content ratio of the structural unit (2) may be measured in the same manner as the content ratio of the structural unit (1).

Also, in a case where the content ratio of the structural unit (2) is 30 mass % or less and n in Formula (2) is 35 or less in terms of an average value, particularly in a case where the content ratio of the structural unit (2) is 10 mass % or more and 30 mass % or less and n in Formula (2) is 10 to 35 in terms of an average value, the printing performance and the ease of culture are particularly favorable.

The content ratio [(1):(2)] of the structural unit (1) and the structural unit (2) contained in the polymer of the component (A) is preferably 50:50 to 95:5, more preferably 58:42 to 92:8, and particularly preferably 68:32 to 89:11 in terms of a mass ratio in order to obtain desired viscosity characteristics before and after application of stress, to improve printing performance, to enhance ease of culture, and to satisfy having ease of production.

In a case where the content ratio [(1):(2)] is 68:32 or more, printing performance and ease of culture are particularly favorable.

Further, in a case where the content ratio [(1):(2)] is 68:32 or more and n in Formula (2) is 35 or less in terms of an average value, and in a case where the content ratio [(1):(2)] is 68:32 to 89:11 and n in the Formula (2) is 10 to 35 in terms of an average value, the printing performance and ease of culture are particularly favorable.

The polymer of the component (A) preferably has a structural unit derived from a crosslinkable monomer in addition to the structural unit (1) in order to reduce stringiness and obtain a desired viscosity before and after application of stress. The polymer of the component (A) preferably has both the structural unit (2) and a structural unit derived from a crosslinkable monomer, in addition to the structural unit (1).

Examples of the structural unit derived from the crosslinkable monomer include one or more selected from the group consisting of a structural unit derived from a vinyl-based crosslinkable monomer, a structural unit derived from an allyl-based crosslinkable monomer, a structural unit derived from a (meth)acrylate-based crosslinkable monomer, and a structural unit derived from a (meth)acrylamide-based crosslinkable monomer. Also, the crosslinkable monomer is preferably a di- to penta-functional crosslinkable monomer, and more preferably a di to tetra-functional crosslinkable monomer. Among the crosslinkable monomers, an allyl-based crosslinkable monomer and a (meth)acrylate-based crosslinkable monomer are preferable, and an allyl-based crosslinkable monomer is more preferable in order to obtain high visibility. In addition, the crosslinkable monomer is preferably a nonionic crosslinkable monomer in order to improve salt tolerance.

Also, the crosslinkable monomer may be preferably a crosslinkable monomer having a degradable partial structure in some cases. Examples of the degradable partial structure include a disulfide bond, an ester bond, a thioester bond, an acetal bond, a benzyl ester structure, and a nitrobenzyl structure. As an example, in a case where a crosslinkable monomer having a disulfide bond is used and a reducing agent such as dithiothreitol or glutathione is added, a three-dimensional structure can be easily obtained from a container. The crosslinkable monomer having a degradable partial structure is preferably an allyl-based crosslinkable monomer having a degradable partial structure because of high availability.

Examples of the vinyl-based crosslinkable monomer include aromatic vinyl-based crosslinkable monomers such as divinylbenzene, trivinylbenzene, divinyltoluene, divinylxylene, and divinylethylbenzene; N,N′-alkylene bis(N-vinylacetamide) such as N,N′-methylene bis(N-vinylacetamide), N,N′-ethylene bis(N-vinylacetamide), N,N′-propylene bis(N-vinylacetamide), N,N′-butylene bis(N-vinylacetamide), and N,N′-hexylene bis(N-vinylacetamide); N,N′-alkylene bis(N-vinylformamide) such as N,N′-butylene bis(N-vinylformamide), and also include divinyl ether and N,N′-(diacetyl)-N,N′-(divinyl)-1,3-bis(aminomethyl) cyclohexane. These can be used singly or in combination of two or more kinds thereof.

Examples of the allyl-based crosslinkable monomer include pentaerythritol diallyl ether, pentaerythritol triallyl ether, pentaerythritol tetraallyl ether, tetraallyloxyethane, triallyl phosphate, trimethylolpropane diallyl ether, trimethylolpropane triallyl ether, allylcio sugar, diallyl phthalate, diallyl isophthalate, diallyl terephthalate, diallyl maleate, diallyl fumarate, diallyl itaconate, triallyl trimellitate, diallyl disulfide, and bis(1,3-bis(allyloxy)propane-2-yl)disulfide. These can be used singly or in combination of two or more kinds thereof.

Examples of the (meth)acrylate-based crosslinkable monomer include ethylene glycol di(meth)acrylate, diethylene glycol di(meth)acrylate, triethylene glycol di(meth)acrylate, tetraethylene glycol di(meth)acrylate, polyethylene glycol di(meth)acrylate, propylene glycol di(meth)acrylate, dipropylene glycol di(meth)acrylate, tripropylene glycol di(meth)acrylate, tetrapropylene glycol di(meth)acrylate, polypropylene glycol di(meth)acrylate, 1,4-butanediol di(meth)acrylate, 1,6-hexanediol di(meth)acrylate, glycerin di(meth)acrylate, trimethylolethane di(meth)acrylate, trimethylolpropane di(meth)acrylate, trimethylolpropane tri(meth)acrylate, butane triol di(meth)acrylate, pentaerythritol di(meth)acrylate, pentaerythritol tri(meth)acrylate, pentaerythritol tetra(meth)acrylate, glucose di(meth)acrylate, glucose tri(meth)acrylate, glucose tetra(meth)acrylate, dipentaerythritol di(meth)acrylate, dipentaerythritol tri(meth)acrylate, dipentaerythritol tetra(meth)acrylate, dipentaerythritol penta(meth)acrylate, inositol di(meth)acrylate, inositol tri(meth)acrylate, inositol tetra(meth)acrylate, mannitol di(meth)acrylate, mannitol tri(meth)acrylate, mannitol tetra(meth)acrylate, mannitol penta(meth)acrylate, and bis(2-(meth)acryloyl)oxyethyl disulfide. These can be used singly or in combination of two or more kinds thereof.

Examples of the (meth)acrylamide-based crosslinkable monomer include N,N′-methylene bis(meth)acrylamide, N,N′-ethylene bis(meth)acrylamide, N,N′-propylene bis(meth)acrylamide, N,N′-butylene bis(meth)acrylamide, N,N′-hexylene bis(meth)acrylamide, and N,N′-bis(((meth)acryloyl)cystamine. These can be used singly or in combination of two or more kinds thereof.

A content ratio of the structural unit (hereinafter, also referred to as “structural unit (3)”) derived from the crosslinkable monomer is preferably 0.1 mass % or more, more preferably 0.5 mass % or more, still more preferably 1 mass % or more, still further preferably 2 mass % or more, and particularly preferably 2.5 mass % or more with respect to the total structural units in the polymer of the component (A) in order to reduce stringiness and obtain a desired viscosity before and after application of stress, and is preferably 10 mass % or less, more preferably 7 mass % or less, and particularly preferably 5 mass % or less with respect to the total structural units in the polymer of the component (A) in order to maintain a high swelling degree and obtain a high viscosity. A specific range is preferably 0.1 mass % or more and 10 mass % or less, more preferably 0.5 mass % or more and 7 mass % or less, still more preferably 1 mass % or more and 7 mass % or less, still further preferably 2 mass % or more and 7 mass % or less, and particularly preferably 2.5 mass % or more and 5 mass % or less, with respect to all structural units in the polymer of the component (A).

The content ratio of the structural unit (3) may be measured in the same manner as the content ratio of the structural unit (1).

The content ratio [(1):(3)] of the structural unit (1) and the structural unit (3) contained in the polymer of the component (A) is preferably 83:17 to 99.99:0.01, more preferably 89:11 to 99.9:0.1, still more preferably 93:7 to 99:1, particularly preferably 94:6 to 99:1 in terms of a mass ratio in order to increase transparency (visibility at the time of printing or cell culture) and to adjust the degree of swelling to obtain high viscosity.

In a case where the content ratio [(1):(3)] is 94:6 or more, the transparency (visibility at the time of printing or cell culture) is particularly good.

The polymer of the component (A) may have a structural unit other than the structural unit (1), the structural unit (2), and the structural unit derived from the crosslinkable monomer. Examples thereof include a structural unit derived from a non-crosslinkable monomer other than the structural unit (1) and the structural unit (2), and a structural unit derived from a nonionic non-crosslinkable monomer is preferable. Specific examples of such a non-crosslinkable monomer include methyl(meth)acrylate, ethyl(meth)acrylate, hydroxymethyl(meth)acrylate, 2,3-dihydroxypropyl(meth)acrylate, (meth)acrylamide, N-methyl(meth)acrylamide, N,N-dimethyl(meth)acrylamide, N-hydroxymethyl(meth)acrylamide, N-hydroxyethyl(meth)acrylamide, N-methoxymethyl(meth)acrylamide, N-ethoxymethyl(meth)acrylamide, polyethylene glycol mono(meth)acrylamide, methoxypolyethylene glycol(meth)acrylamide, polypropylene glycol mono(meth)acrylamide, methoxypolypropylene glycol(meth)acrylamide, allyl alcohol, allyl methyl ether, allyl ethyl ether, ethylene glycol monoallyl ether, pentaerythritol monoallyl ether, and polyethylene glycol monoallyl ether.

Examples of the polymer of the component (A) include a particulate polymer, a monolithic polymer, a plate-like polymer, a film-like polymer, a fibrous polymer, and a chip-like polymer, and a particulate polymer is preferable, and a gel particulate polymer is more preferable, in order to reduce stringiness and obtain a desired viscosity before and after application of stress.

The polymer of the component (A) is preferably a nonionic polymer in order to improve salt tolerance.

In a case where the polymer of the component (A) is a particulate polymer, the volume average particle size is preferably 0.05 to 100 μm and more preferably 0.1 to 50 μm. The coefficient of variation of the volume average particle diameter is preferably 30% or less, and more preferably 25% or less.

The volume average particle size and the coefficient of variation can be measured by atomic force microscope observation in liquid, phase contrast microscope observation, laser diffraction/scattering particle size distribution measurement, or the like. In addition, after the particles are fluorescently stained, measurement can be performed by confocal laser microscope observation or the like.

In the polymer as the component (A), when the viscosity of the dispersion is measured under the conditions of a measurement temperature of 23° C. and a shear rate of 100 sec⁻¹ using a rotary viscometer (for example, a rotary viscometer ViscoQC 300R manufactured by Anton Paar GmbH) while dispersing the polymer in pure water, the content of pure water at which the viscosity of the dispersion reaches a value smaller than 1000 mPa·s is preferably 70 mass % or more, more preferably 80 mass % or more, still more preferably 90 mass % or more, and particularly preferably 95 mass % or more. Also, the content is usually 99.9 mass % or less.

The pure water content may be measured in accordance with the method described in Examples described later.

The polymer of the component (A) can be produced by appropriately combining known methods described in JP 10-226715 A, JP 2002-239380 A, and the like.

The content ratio of the polymer of the component (A) is preferably 0.1 mass % or more, more preferably 0.2 mass % or more, and particularly preferably 0.5 mass % or more in the composition of the present invention in order to enhance the shape maintainability of the three-dimensional structure, and is preferably 30 mass % or less, more preferably 20 mass % or less, and particularly preferably 10 mass % or less in the composition of the present invention in order to enhance the ease of three-dimensional culture and to improve visibility. A specific range thereof is preferably 0.1 mass % or more and 30 mass % or less, more preferably 0.2 mass % or more and 20 mass % or less, and particularly preferably 0.5 mass % or more and 10 mass % or less in the composition of the present invention.

### (Component (B))

The composition of the present invention contains (B) an aqueous medium.

Examples of the aqueous medium include one or more selected from the group consisting of water, alcohol, and a culture medium, and water, a culture medium, a mixed solution of water and alcohol, and a mixed solution of water and a culture medium are preferable. In a case where the composition of the present invention is used for a 3D cell culture support, the component (B) is particularly preferably a culture medium or a mixed solution of water and a culture medium.

As the alcohol, a lower alcohol is preferable, and a linear or branched monohydric alcohol having 1 to 6 carbon atoms is more preferable. Examples thereof include ethanol, isopropanol, and n-propanol. These can be used singly or in combination of two or more kinds thereof.

A content ratio of water in the mixed solution of water and alcohol is preferably 80 mass % or more and less than 100 mass %, and more preferably 90 mass % or more and less than 100 mass % in order to act as a stable culture medium when used for cell culture.

The culture medium is not particularly limited as long as the cells can survive or grow, and examples thereof include Eagle's medium, Ham's medium, Fisher's medium, Dulbecco's modified MEM (DMEM) medium, MEM medium, F12 medium, RPMI1640 medium, MCDB104 medium, 199 medium, MCDB153 medium, L15 medium, SkBM medium, Basal medium, and a medium containing a mixed medium thereof.

The culture medium may be either a serum medium or a serum-free medium. Examples of the serum include fetal bovine serum. In addition, for example, minerals, a carbon source (such as glucose and carbon dioxide), a nitrogen source (such as glutamine), antibiotics, a vitamin source, a mineral source, proteins, peptides, may be added to the culture medium. As the minerals, a multivalent ion source is preferable. Examples of the multivalent ion source include calcium ion sources such as calcium carbonate, calcium hydrogen phosphate, and calcium chloride. According to the present invention, even in a case where such a multivalent ion source is used as minerals, three-dimensional cell culture can be efficiently performed. The culture medium used in the present invention may be a culture medium containing the plurality of culture media or additives.

The content ratio of the aqueous medium of the component (B) is preferably 70 mass % or more, more preferably 80 mass % or more, and particularly preferably 90 mass % or more in the composition of the present invention in order to enhance ease of three-dimensional culture and improve visibility, and is preferably 99.9 mass % or less, more preferably 99.8 mass % or less, and particularly preferably 99.5 mass % or less in the composition of the present invention in order to enhance shape maintainability of a three-dimensional structure. A specific range thereof is preferably 70 mass % or more and 99.9 mass % or less, more preferably 80 mass % or more and 99.8 mass % or less, and particularly preferably 90 mass % or more and 99.5 mass % or less in the composition of the present invention.

The content ratio [(A):(B)] of the polymer of the component (A) and the aqueous medium of the component (B) contained in the composition of the present invention is preferably 0.1:99.9 to 30:70, more preferably 0.2:99.8 to 20:80, and particularly preferably 0.5:99.5 to 10:90 in terms of a mass ratio in order to achieve both shape maintainability of a three-dimensional structure and ease of three-dimensional culture.

The composition of the present invention can contain components other than the above (hereinafter, also referred to as other components) as necessary. Examples of the other components include a surfactant, an isotonizing agent (for example, sodium chloride), a chelating agent, a pH adjusting agent, a buffering agent, a thickening agent, and a stabilizing agent. These can be used singly or in combination of two or more kinds thereof.

As the composition of the present invention, a plastic fluid composition is preferable. Here, the “plastic fluid” is a kind of non-Newtonian fluid, and means a fluid that does not flow until a certain shear stress (yield stress) is applied. Also, as the composition of the present invention, a plastic fluid composition is preferable. In the present specification, the slurry refers to a mixture of a solid substance in a liquid.

In the composition of the present invention, the viscosity measured under the conditions of a measurement temperature of 23° C. and a shear rate of 100 sec⁻¹ using a rotary viscometer (for example, a rotary viscometer ViscoQC 300R manufactured by Anton Paar GmbH) is preferably 1 mPa·s or more and 1×10⁵ mPa·s or less, more preferably 5 mPa·s or more and 5×10⁴ mPa·s or less, still more preferably 10 mPa·s or more and 1×10⁴ mPa·s or less, particularly preferably 50 mPa·s or more and 5×10³ mPa·s or less. The viscosity measured under the conditions of a measurement temperature of 23° C. and a shear rate of 0.1 sec⁻¹ is preferably 1×10³ mPa·s or more and 1×10⁸ mPa·s or less, more preferably 5×10³ mPa s or more and 5×10⁷ mPa s or less, still more preferably 1×10⁴ mPa s or more and 1×10⁷ mPa·s or less, particularly preferably 5×10⁴ mPa·s or more and 5×10⁶ mPa·s or less.

The viscosity can be determined in accordance with JIS Z 8803:2011. Specifically, measurement may be performed in accordance with a method described in examples which will be described later.

In the composition of the present invention, a ratio (X/Y) between the viscosity X measured under the conditions of a measurement temperature of 23° C. and a shear rate of 0.1 sec⁻¹ using a rotary viscometer (for example, a rotary viscometer ViscoQC 300R manufactured by Anton Paar GmbH) and the viscosity Y measured under the conditions of a measurement temperature of 23° C. and a shear rate of 100 sec⁻¹ using the same viscometer is preferably 10 or more, more preferably 50 or more, still more preferably 75 or more, and particularly preferably 100 or more. Also, the ratio is usually 10000 or less. A higher ratio indicates that it has desirable viscosity properties like plastic fluid.

In the composition of the present invention, the viscosity change rate when NaCl is added, calculated in accordance with the following formula (α), is preferably −50% or more and 50% or less, more preferably −30% or more and 20% or less, still more preferably −20% or more and 10% or less, particularly preferably −10% or more and 5% or less.

$$Viscosity change rate ( \% ) = \{ ( Viscosity before addition of NaCl ) - ( Viscosity after addition of NaCl ) \} / ( Viscosity before addition of NaCl ) \times 100 \qquad ( α )$$

[In Formula (α), the viscosity before addition of NaCl means a viscosity (mPa·s) of the composition when measured under the conditions of a measurement temperature of 23° C. and a shear rate of 100 sec⁻¹ using a rotary viscometer (for example, a rotary viscometer ViscoQC 300R manufactured by Anton Paar GmbH), and the viscosity after addition of NaCl means a viscosity (mPa·s) of a composition obtained by adding NaCl to the composition so that a NaCl concentration is 0.15 mol/L and then allowing the composition to stand for 60 minutes, measured under the conditions of a measurement temperature of 23° C. using a rotary viscometer.]

The viscosity change rate may be measured in accordance with the method described in Examples described later. The viscosity of the composition of the present invention after addition of NaCl when measured under the conditions of a measurement temperature of 23° C. and a shear rate of 100 sec⁻¹ is preferably 1 mPa·s or more and 1×10⁵ mPa·s or less, more preferably 5 mPa·s or more and 5×10⁴ mPa·s or less, still more preferably 10 mPa s or more and 1×10⁴ mPa s or less, and particularly preferably 50 mPa s or more and 5×10³ mPa·s or less.

### (Composition (II))

The present invention also provides a composition for a 3D printing support or a 3D cell culture support, containing the following components (A2) and (B).

(A2) Nonionic polymer in which a pure water content at which a viscosity of a dispersion reaches a value less than 1000 mPa·s when the viscosity of the dispersion is measured using a rotary viscometer (for example, a rotary viscometer ViscoQC 300R manufactured by Anton Paar GmbH) under the conditions of a measurement temperature of 23° C. and a shear rate of 100 sec⁻¹ while dispersing the dispersion in pure water is 70 mass % or more

### (B) Aqueous Medium

In the polymer as the component (A2), when the viscosity of the dispersion is measured under the conditions of a measurement temperature of 23° C. and a shear rate of 100 sec⁻¹ using a rotary viscometer (for example, a rotary viscometer ViscoQC 300R manufactured by Anton Paar GmbH) while dispersing the polymer in pure water, the content of pure water at which the viscosity of the dispersion reaches a value smaller than 1000 mPa·s is preferably 70 mass % or more, more preferably 80 mass % or more, still more preferably 90 mass % or more, and particularly preferably 95 mass % or more. Also, the content is usually 99.9 mass % or less.

The pure water content may be measured in accordance with the method described in Examples described later.

As the polymer of the component (A2), a polymer having the structural unit (1) is preferable. The structural unit (1) is the same as that of the polymer of the component (A).

The polymer of the component (A2) is preferably the same as the polymer of the component (A). The composition (II) of the present invention is preferably the same as the composition of the present invention. The aqueous medium (B) contained in the composition (II) of the present invention is the same as the aqueous medium (B) contained in the composition of the present invention.

The composition of the present invention and the composition (II) of the present invention are useful for a 3D printing support or a 3D cell culture support. In addition, it is excellent in 3D printing performance and ease of three-dimensional cell culture, is hardly affected by impurities when used for 3D printing or three-dimensional cell culture, and is also suitable for manufacturing a complicated and flexible three-dimensional structure such as a biological tissue or an organ. Furthermore, since transparency is high, visibility at the time of printing or cell culture is high.

The composition of the present invention and the composition (II) of the present invention are excellent in salt tolerance. Therefore, even when a salt or ions are added, the viscosity is less likely to decrease, and when a multivalent ion source (calcium ion source or the like) is added using the multivalent ion source as a 3D cell culture support, ions easily reach cells, and cells are easily grown. It can also be used for 3D printing using inks that cure in response to calcium ions, for example.

Here, the “3D printing support” means a material that is used for 3D printing in which a desired structure can be created in a three-dimensional space, and is configured to be able to be arranged so as to embed a structure material, thereby supporting the structure material in the three-dimensional space. In addition, the “3D cell culture support” means a material that is used for 3D cell culture in which desired cells can be cultured in a three-dimensional space, is arranged so as to embed a composition containing the desired cells when the desired cells are cultured in the three-dimensional space, and supports the composition containing the desired cells in the three-dimensional space. Note that “for a 3D printing support or for a 3D cell culture support” is a concept including those used for both a 3D printing support and a 3D cell culture support.

### (Method for Manufacturing Three-Dimensional Structure)

The three-dimensional structure manufacturing method of the present invention includes the following step (i) and step (ii). The three-dimensional structure manufacturing method of the present invention is similar to the known 3D printing method and 3D bioprinting method except that the composition of the present invention or the composition (II) of the present invention is used. For example, it can be produced by an inkjet method, a material extrusion method, a stereolithography method, or the like. Specifically, it may be performed with reference to the description of WO2018/187595, WO2018/165584, WO2015/129881, and the like.
(i) Step of filling a container with the composition of the present invention or the composition (II) of the present invention
(ii) Step of bringing a second composition into contact with the composition with which the container is filled in step (i)

### (Step (i))

Examples of the container used in the step (i) include a glass container, a plastic container, a metal container, and a plastic container is preferable because the state of 3D printing can be confirmed and the cell culture can be performed as it is. Also, semitransparent or a transparent container is preferable.

(Step (ii))

The second composition may be any composition as long as the composition acts as an ink or a bioink in 3D printing or three-dimensional cell culture, and examples thereof include curable compositions in addition to those containing a cell and an aqueous medium.

In a case where a composition containing a cell and an aqueous medium is used as the second composition, three-dimensional cells can be produced as a three-dimensional structure. Examples of the three-dimensional cell include an organoid, a spheroid, an embryoid body, a tumor, a cyst, and a microtissue, and the three-dimensional cell is preferably an organoid or a spheroid, and particularly preferably an organoid. The three-dimensional structure manufacturing method of the present invention is suitable for manufacturing a complicated and flexible three-dimensional structure such as a living tissue or an organ, and particularly suitable for manufacturing an organoid.

Examples of the cells include anchorage-dependent cells and floating cells (for example, blood cells such as white blood cells, red blood cells, and platelets). Examples of the anchorage-dependent cells include HeLa cells and cancer cells such as F9 cells; fibroblasts such as 3T3 cells; stem cells such as ES cells, iPS cells, and mesenchymal stem cells; kidney cells such as HEK293 cells; neurons such as NT2 cells; endothelial cells such as UV♀2 cells, HMEC-1 cells, and HUVEC; cardiomyocytes such as H9c2 cells; and epithelial cells such as Caco-2 cells.

Examples of the aqueous medium include one or more selected from the group consisting of water, alcohol, and a culture medium. In addition, for example, a surfactant, an isotonizing agent (for example, sodium chloride), a chelating agent, a pH adjusting agent, a buffering agent, a thickener, a stabilizer, may be further contained.

In a case where a composition containing cells and an aqueous medium is used as the second composition, the second composition preferably further contains at least one selected from the group consisting of an extracellular matrix and a hydrogel, and more preferably contains an extracellular matrix.

Examples of the extracellular matrix component include a component contained in the basement membrane and a glycoprotein present in the intercellular space. Examples of the component contained in the basement membrane include type IV collagen, laminin, heparan sulfate proteoglycan, and entactin. Examples of the glycoprotein present in the intercellular space include collagen, laminin, entactin, fibronectin, fibrinogen, and heparin sulfate. These can be used singly or in combination of two or more kinds thereof.

Examples of the hydrogel include a combination of a polysaccharide and, if necessary, a coagulant corresponding to the polysaccharide. Examples of the polysaccharide include hyaluronic acid, hyaluronic acid salt, alginic acid, alginic acid salt, carrageenan, glucomannan, agarose, cellulose, pectin, gellan gum, chitin, chitosan, chondroitin sulfate, and polysaccharides obtained by subjecting a natural product to a hydrolysis treatment with an acid or a base, or a chemical modification treatment such as acetylation can also be used. These can be used singly or in combination of two or more kinds thereof. Examples of the coagulant include divalent metal salts. Examples of the divalent metal salt include a barium salt, a calcium salt, and a magnesium salt. According to the present invention, even in a case where such a divalent metal salt is used as a coagulant, three-dimensional cell culture can be efficiently performed.

In a case where the curable composition is used as the second composition, a 3D printing shaped object can be manufactured as a three-dimensional structure. Examples of such a shaped object include a model for design image, an industrial component, and a medical device.

Examples of the curable composition include, in addition to the extracellular matrix component and the hydrogel, thermoplastic resins such as ABS resin, polyethylene, polypropylene, vinyl chloride resin, polyethylene terephthalate, polycarbonate, polyacetal, and polyimide; thermosetting resins such as phenol resin, epoxy resin, melamine resin, and silicone resin; photocurable resin such as acrylic resin; and compositions containing inorganic substances such as silica and hydroxyapatite. These can be used singly or in combination of two or more kinds thereof.

In addition, a monomer, an oligomer, a reaction initiator, a solvent, and the like may be further contained. These can be used singly or in combination of two or more kinds thereof.

The step (ii) is preferably a step of injecting the second composition into the composition with which the container is filled in the step (i) while applying a shear. In a case where the force applied by shearing is less than or equal to the yield value of the composition of the present invention or the composition (II) of the present invention, the composition of the present invention or the composition (II) of the present invention with which the container is filled is solid or semi-solid, but in a case where the force applied by shearing is greater than the yield value of the composition of the present invention or the composition (II) of the present invention, the composition of the present invention or the composition (II) of the present invention with which the container is filled becomes liquid. In this case, the drawing (stress application) is performed by injecting the second composition into the composition of the present invention or the composition (II) of the present invention. In addition, in a case where the applied stress is removed, the composition of the present invention or the composition (II) of the present invention becomes solid or semi-solid again.

The shearing may be applied by any energy such as mechanical, electrical, radiation, or light.

Also, the second composition is injected through, for example, an injector, a dispenser, a microchannel, or the like. In a case where three-dimensional cells are produced, injection may be performed with a syringe, a pipette, or an automatic cell injector. For example, in a case of using a computer-controlled cell injector, the second composition containing a cell and an aqueous medium is injected at a plurality of locations along the path forming the desired 3D pattern while applying shear to the composition of the present invention or the composition of the present invention (II) with which a container is filled.

In this manner, by bringing the second composition into contact with the composition with which the container is filled in the step (i) (preferably, injecting the second composition while applying shear), a three-dimensional structure having a desired shape can be produced.

In particular, in a case where a composition containing a cell and an aqueous medium is used as the second composition, the cell can be disposed at a desired position. When the second composition is not used, cells or the like are directly contained in the composition of the present invention, and a container is filled with this composition, cells can be placed although the desired shape and position as described above are not obtained.

In addition, in a case where a composition containing a cell and an aqueous medium is used as the second composition, it is preferable to culture the injected cells after injecting the second composition. According to this, the cells adhere, extend and proliferate to form three-dimensional cells.

In addition, the obtained three-dimensional structure can be recovered according to a conventional method. In a case where a composition containing a cell and an aqueous medium is used as the second composition, the three-dimensional cells can be collected by suction with, for example, a syringe or a pipette.

According to the three-dimensional structure manufacturing method of the present invention, the three-dimensional structure can be easily and efficiently manufactured. The three-dimensional structure manufacturing method of the present invention is hardly affected by impurities during 3D printing (drawing) or three-dimensional cell culture, and is also suitable for manufacturing a complicated and flexible three-dimensional structure such as a biological tissue or an organ.

In addition, the obtained three-dimensional cells can be used, for example, for evaluation of toxicity and drug efficacy of substances, elucidation of biochemical functions of cells, search for biomarkers, and the like.

### EXAMPLES

Hereinafter, the present invention will be described in detail with reference to Examples, but the present invention is not limited to these Examples.

### Example 1

(1) 270 g of ethyl acetate (manufactured by FUJIFILM Wako Pure Chemical Corporation), 21.6 g of N-vinylacetamide (manufactured by Showa Denko K.K.), 7.5 g of M-230G (manufactured by Shin Nakamura Chemical Co., Ltd.) as methoxy polyethylene glycol(23)monomethacrylate, and 0.9 g of pentaerythritol allyl ether ((mixture, as a prime component, containing pentaerythritol diallyl ether, pentaerythritol triallyl ether, and pentaerythritol tetraallyl ether) manufactured by Sigma-Aldrich Co. LLC.) was charged into a separable flask, and stirred to be dissolved. A temperature of the resulting product was increased to 65° C., and 0.3 g of 2,2′-azoisobutyronitrile (manufactured by Wako Pure Chemical Industries, Ltd.) was added thereto while stirring under a nitrogen atmosphere, to initiate polymerization. Stirring was continued for 6 hours while maintaining the temperature. The resulting polymer was filtered and washed with ethyl acetate, and then dried under reduced pressure to produce a polymer A1.

(2) Thereafter, 10 g of the polymer A1 was put in 190 g of pure water in a polypropylene container and dispersed therein to produce a composition B1 for a support.

### Example 2

A polymer A2 and a composition B2 for a support were produced in the same manner as in Example 1 except that the amount of N-vinylacetamide used was changed to 17.8 g and the amount of M-230G used was changed to 11.3 g.

### Example 3

A polymer A3 and a composition B3 for a support were produced in the same manner as in Example 1 except that M-230G was changed to M-450G (manufactured by Shin Nakamura Chemical Co., Ltd.) as methoxy polyethylene glycol(45)monomethacrylate.

### Example 4

A polymer A4 and a composition B4 for a support were produced in the same manner as in Example 1 except that the amount of N-vinylacetamide used was changed to 21.0 g and the amount of pentaerythritol allyl ether used was changed to 1.5 g.

### Comparative Example 1

(1) 270 g of ethyl acetate, 29.7 g of acrylic acid (manufactured by FUJIFILM Wako Pure Chemical Corporation), and 0.3 g of pentaerythritol allyl ether was charged into a separable flask and stirred to be dissolved. A temperature of the resulting product was increased to 65° C., and 0.3 g of 2,2′-azoisobutyronitrile (manufactured by Wako Pure Chemical Industries, Ltd.) was added thereto while stirring under a nitrogen atmosphere, to initiate polymerization. Stirring was continued for 6 hours while maintaining the temperature. The resulting polymer was dried under reduced pressure to produce a polymer A′1.

(2) Thereafter, 2 g of the polymer A′1 was put in 198 g of pure water in a polypropylene container, dispersed therein, and then neutralized with a 5 mol/L aqueous solution of sodium hydroxide (manufactured by FUJIFILM Wako Pure Chemical Corporation) to pH7, thereby producing a composition B′1 for a support.

### Test Example 1 (Measurement of Viscosity)

In accordance with JIS Z 8803:2011, the viscosity (mPa·s) of the composition for a support was measured under the conditions of 23° C. and a shear rate of 100 s⁻¹ using a rotary viscometer ViscoQC 300R and an adapter jig CC12 (both of which are manufactured by Anton Paar).

### Test Example 2 (Measurement of Water Content at Viscosity of Less than 1000 mPa·s)

Each composition for a support was diluted with pure water in such a way that the water content increased by 0.1 mass %, and the viscosity at each dilution was measured until the viscosity value became less than 1000 mPa·s. The water content (mass %) when the viscosity was less than 1000 mPa·s was recorded. The results are shown in Table 1. In a case where the water content when the viscosity is less than 1000 mPa·s is 90 mass % or more, it can be said that the polymer easily absorbs water and is hardly affected by impurities when the composition for a support is used for 3D printing or 3D cell culture.

The viscosity was measured in accordance with the conditions of Test Example 1. In a case where the water content was 99 mass % or more, dilution was performed with pure water in such a way that the water content increased by 0.05 mass %, and the viscosity was measured in the same manner.

In addition, a sample diluted to immediately before reaching the viscosity of less than 1000 mPa s obtained in this test was used as a composition for a support in Test Examples 3 to 7.

### Test Example 3 (Evaluation of Shear Rate Dependency of Viscosity)

The shear rate dependency of the viscosity was evaluated for the composition for a support diluted to immediately before reaching the viscosity of less than 1000 mPa·s in Test Example 2.

That is, the viscosity of the composition for a support was measured under the conditions of 23° C. and a shear rate of 0.1 (1/s) using a rotary viscometer ViscoQC 300R and an adaptor jig CC18 (both of which are manufactured by Anton Paar), and the resulting viscosity value was defined as X. Furthermore, the adapter jig was changed to CC12 manufactured by Anton Paar, and the viscosity was measured under conditions of 23° C. and a shear rate of 100 (1/s), and the resulting viscosity value was designated as Y. The ratio (X/Y) of the viscosity value X to the viscosity value Y was determined, and the printing performance was evaluated in accordance with the following criteria. The results are shown in Table 1.

### (Printing Performance Evaluation Criteria)

AAA (better than AA): 100 or more
AA (better than A): 75 or more and less than 100
A (good): 50 or more and less than 75

### Test Example 4 (Evaluation of Transparency)

For the composition for a support diluted to immediately before reaching the viscosity of less than 1000 mPa·s in Test Example 2, the absorbance at a wavelength of 600 nm and an optical path length of 10 mm was measured using a spectrophotometer BioSpectrometer (manufactured by Eppendorf). The results are shown in Table 1. As shown in Table 1, the composition for a support of Examples 1 to 4 had high transparency. The composition for a support of Examples 1 to 3 had particularly high transparency and high visibility at the time of printing or cell culture.

### Test Example 5 (Evaluation of Salt Tolerance)

Sodium chloride (manufactured by FUJIFILM Wako Pure Chemical Corporation) was added to 40 g of the composition for a support diluted to immediately before reaching the viscosity of less than 1000 mPa·s in Test Example 2 in such a way that the final concentration was 0.15 mol/L, and the mixture was stirred to be dissolved. The viscosity was measured 60 minutes after the addition of sodium chloride in accordance with the conditions of Test Example 1. The viscosity change rate (%) was calculated in accordance with the following Formula (α), provided that the viscosity (≈1000 mPa·s) at less than 1000 mPa·s measured in Test Example 2 is defined as “viscosity before addition of NaCl”. The results are shown in Table 1. In a case where the viscosity change rate is 20% or less, it can be said that salt tolerance is excellent.

$$Viscosity change rate ( \% ) = \{ ( Viscosity before addition of NaCl ) - ( Viscosity after addition of NaCl ) \} / ( Viscosity before addition of NaCl ) \times 100 \qquad ( α )$$

### Test Example 6 (Evaluation of 3D Printing Performance)

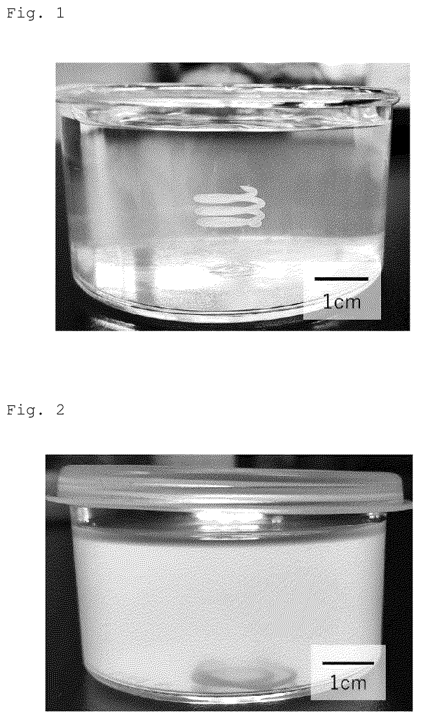

To 100 g of the composition for a support diluted to immediately before reaching the viscosity of less than 1000 mPa·s in Test Example 2, sodium chloride (manufactured by FUJIFILM Wako Pure Chemical Corporation) was added in such a way that the final concentration was 0.15 mol/L, and calcium chloride (manufactured by FUJIFILM Wako Pure Chemical Corporation) was added in such a way that the final concentration was 10 mmol/L, respectively, and the resulting mixture was stirred and dissolved to prepare a calcium ion-containing 3D printing support. A 3D bioprinter INKREDIBLE (manufactured by Cellink) was used to make test patterns in the calcium ion-containing 3D printing support using CELLINK XPLORE (manufactured by Cellink) as an ink that cures in response to calcium ions. The shape maintainability and the cured state of the test pattern 1 hour after making were evaluated in accordance with the following criteria. The results are shown in Table 1. In addition, FIG. 1 shows a test pattern in a case where the composition for a support of Example 1 was used, and FIG. 2 shows a test pattern in a case where the composition for a support of Comparative Example 1 was used. As shown in FIG. 1, the transparency of the bath was high in a case where the composition for a support of Example 1 was used, whereas the turbidity was observed in a case where the composition for a support of Comparative Example 1 was used. It considered that the reason why the turbidity occurs in the composition for a support of Comparative Example 1 is that a salt such as calcium chloride was added.

### (Shape Maintainability)

A: The test pattern does not sink, and the shape is completely maintained.
B: Although sedimentation of the test pattern is observed, the shape is basically maintained.
C: Sedimentation of the test pattern was observed, and the shape was collapsed.

### (Cured State)

A: Curing of the test pattern is sufficient, and the test pattern can be taken out without being broken.
B: Curing of the test pattern is insufficient, and only a part of the test pattern can be taken out.
C: Curing of the test pattern was insufficient, and the test pattern could not be taken out at all.

### Test Example 7 (Evaluation of Cell Culture)

100 g of the composition for support diluted to immediately before reaching the viscosity of less than 1000 mPa·s in Test Example 2 was sterilized in an autoclave, and 1.34 g of Dulbecco's modified Eagle's medium powder (manufactured by Sigma-Aldrich Co. LLC.) and 11 mL of fetal bovine serum (manufactured by Sigma-Aldrich Co. LLC.) were added and mixed uniformly. HEK293 cells were mixed therein so as to have a concentration of 1.0×10⁵/mL, and 2 mL of the resulting mixture was added to a 12 well plate. After air bubbles were removed by centrifugation at 800×g for 1 minute, the cells were cultured for 72 hours in an incubator with a carbon dioxide concentration of 5%. After culture, the total amount of cells was collected using 10 mL of PBS, followed by trypsin treatment, and then the number of living cells was counted by a trypan blue staining method. The proliferation rate of living cells was evaluated in accordance with the following criteria. The results are shown in Table 1.

### (Proliferation Rate of Living Cells)

AA: The number of living cells is 5 times or more the number at the time of seeding
A: The number of living cells is 2 times or more and less than 5 times the number at the time of seeding
B: The number of living cells is less than 2 times the number at the time of seeding

TABLE 1

| | | | | | | Comparative |
|---|---|---|---|---|---|---|
| | | Example 1 | Example 2 | Example 3 | Example 4 | Example 1 |
| Particulate | N-vinylacetamide | 72 | 59.5 | 72 | 70 | 0 |
| polymer | Methoxy polyethylene | 25 | 37.5 | 0 | 25 | 0 |
| composition | glycol(23)monomethacrylate | | | | | |
| (mass %) | Methoxy polyethylene | 0 | 0 | 25 | 0 | 0 |
| | glycol(45)monomethacrylate | | | | | |
| | Pentaerythritol allyl ether | 3 | 3 | 3 | 5 | 1 |
| | Acrylic acid | 0 | 0 | 0 | 0 | 99 |
| Water Content at Less than 1000 mPa · s (mass %) | | 96.1 | 95.3 | 96.4 | 96.3 | 99.70 |
| Printing | Ratio (X/Y) of viscosity value X to | 118 | 81 | 59 | 212 | 78 |
| performance | viscosity value Y | | | | | |
| | Evaluation | AAA | AA | A | AAA | AA |
| Salt tolerance | Viscosity change rate (%) | 1 | 1 | 2 | 0 | 99 |
| Bath evaluation | Transparency of bath | 0.135 | 0.06 | 0.02 | 0.369 | 0.044 |
| | (absorbance) | | | | | |
| | Shape maintainability evaluation | A | A | A | A | C |
| | Cured state evaluation | A | A | A | A | C |
| | Evaluation of Cell Culture | AA | A | A | AA | B |

1. A composition suitable for a 3D printing support or a 3D cell culture support, the composition comprising:
(A) a polymer comprising a structural unit of formula (1): 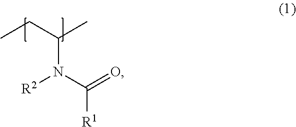
R¹ and R² independently being or an alkyl group comprising 1 to 10 carbon atoms, or R¹ and R² together optionally forming a ring structure comprising 3 to 10 carbon atoms; and
(B) an aqueous medium.

2. The composition of claim 1, wherein the polymer (A) further comprises a structural unit of formula (2), 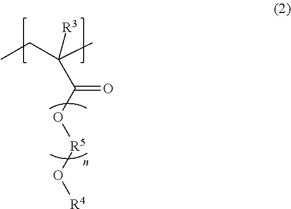
wherein
R³ and R⁴ are independently H or a methyl group,
R⁵ is an alkylene group comprising 2 to 4 carbon atoms, and
n is in a range of from 1 to 1000 in terms of an average value.

3. The composition of claim 1, wherein the polymer (A) further comprises a structural unit derived from a crosslinkable monomer.

4. The composition of claim 3, wherein the structural unit derived from the crosslinkable monomer comprises
a structural unit derived from a vinyl-based crosslinkable monomer,
a structural unit derived from an allyl-based crosslinkable monomer,
a structural unit derived from a (meth)acrylate-based crosslinkable monomer, and/or
a structural unit derived from a (meth)acrylamide-based crosslinkable monomer.

5. The composition of claim 1, wherein the polymer (A) is a particulate polymer.

6. The composition of claim 1, wherein an X/Y ratio, between a viscosity X measured under conditions of a measurement temperature of 23° C. and a shear rate of 0.1 sec⁻¹ using a rotary viscometer and a viscosity Y measured under conditions of a measurement temperature of 23° C. and a shear rate of 100 sec⁻¹ using a rotary viscometer, is 10 or more.

7. The composition of claim 1, wherein a viscosity change rate when NaCl is added, calculated by formula (α), is 20% or less.
$${V}_{CR} = ( {V}_{0} - {V}_{t} ) / {V}_{0} \times 100 , \qquad ( α )$$
wherein V_{CR} is the viscosity change rate in percentage, V₀ is viscosity before addition of NaCl, which is a first viscosity in mPa·s of the composition when measured under a measurement temperature of 23° C. and a shear rate of 100 sec⁻¹ using a rotary viscometer, and V₁ is viscosity after addition of NaCl, which is a second viscosity in mPa·s of a second composition obtained by adding NaCl to the composition in such a way that a resulting NaCl concentration is 0.15 mol/L, and then allowing the second composition to stand for 60 minutes, then measuring the second viscosity under a measurement temperature of 23° C. using a rotary viscometer.

8. A composition suitable for a 3D printing support or a 3D cell culture support, the composition comprising:
(A2) a nonionic polymer in which a pure water content at which a viscosity of a dispersion reaches a value less than 1000 mPa·s, when the viscosity of the dispersion is measured using a rotary viscometer under a measurement temperature of 23° C. and a shear rate of 100 sec⁻¹ while dispersing the dispersion in pure water, is 70 mass % or more; and
(B) an aqueous medium.

9. A method for producing a three-dimensional structure, the method comprising:
(i) filling a container with the composition of claim 1; and
(ii) bringing a second composition into contact with the composition with which the container is filled in the filling (i).

10. The method of claim 9, wherein the bringing (ii) comprises injecting the second composition into the composition with which the container is filled in the filling (i), while applying shear.

11. The method of claim 9, wherein the second composition comprises a cell and an aqueous medium.

12. The method of claim 11, wherein the second composition further comprises an extracellular matrix.

13. The method of claim 9, wherein the three-dimensional structure is an organoid or a spheroid.

14. A three-dimensional structure, obtained by the method of claim 9.

15. The composition of claim 2, wherein the polymer (A) is a particulate polymer.

16. The composition of claim 2, wherein an X/Y ratio, between a viscosity X measured under conditions of a measurement temperature of 23° C. and a shear rate of 0.1 sec⁻¹ using a rotary viscometer and a viscosity Y measured under conditions of a measurement temperature of 23° C. and a shear rate of 100 sec⁻¹ using a rotary viscometer, is 10 or more.

17. The composition of claim 2, wherein a viscosity change rate when NaCl is added, calculated by formula (α), is 20% or less.
$${V}_{CR} = ( {V}_{0} - {V}_{t} ) / {V}_{0} \times 100 , \qquad ( α )$$
wherein V_{CR} is the viscosity change rate in percentage, V₀ is viscosity before addition of NaCl, which is a first viscosity in mPa·s of the composition when measured under a measurement temperature of 23° C. and a shear rate of 100 sec⁻¹ using a rotary viscometer, and Vₜ is viscosity after addition of NaCl, which is a second viscosity in mPa·s of a second composition obtained by adding NaCl to the composition in such a way that a resulting NaCl concentration is 0.15 mol/L, and then allowing the second composition to stand for 60 minutes, then measuring the second viscosity under a measurement temperature of 23° C. using a rotary viscometer.

18. The composition of claim 3, wherein the polymer (A) is a particulate polymer.

19. The composition of claim 3, wherein an X/Y ratio, between a viscosity X measured under conditions of a measurement temperature of 23° C. and a shear rate of 0.1 sec⁻¹ using a rotary viscometer and a viscosity Y measured under conditions of a measurement temperature of 23° C. and a shear rate of 100 sec⁻¹ using a rotary viscometer, is 10 or more.

20. The composition of claim 3, wherein a viscosity change rate when NaCl is added, calculated by formula (α), is 20% or less.
$${V}_{CR} = ( {V}_{0} - {V}_{t} ) / {V}_{0} \times 100 , \qquad ( α )$$
wherein V_{CR} is the viscosity change rate in percentage, V₀ is viscosity before addition of NaCl, which is a first viscosity in mPa·s of the composition when measured under a measurement temperature of 23° C. and a shear rate of 100 sec⁻¹ using a rotary viscometer, and Vₜ is viscosity after addition of NaCl, which is a second viscosity in mPa·s of a second composition obtained by adding NaCl to the composition in such a way that a resulting NaCl concentration is 0.15 mol/L, and then allowing the second composition to stand for 60 minutes, then measuring the second viscosity under a measurement temperature of 23° C. using a rotary viscometer.
